(19) United States
(12) Patent Application Publication
(10) Pub. No.: US 2025/0082641 A1
(43) Pub. Date: Mar. 13, 2025

(54) A PHARMACEUTICAL COMPOSITION COMPRISING COMBINATION OF SGLT2 INHIBITOR AND DPP-IV INHIBITOR

(71) Applicant: UNISON PHARMACEUTICALS PVT. LTD., Ahmedabad (IN)

(72) Inventors: Balvir SINGH, Ahmedabad (IN); Pushpendra SINGH, Ahmedabad (IN); Divyesh KATHROTIA, Ahmedabad (IN); Tejas PATEL, Ahmedabad (IN)

(73) Assignee: UNISON PHARMACEUTICALS PVT. LTD., Ahmedabad (IN)

(21) Appl. No.: 18/580,748

(22) PCT Filed: Jul. 24, 2022
(86) PCT No.: PCT/IN2022/050665

§ 371 Date: Jan. 19, 2024

(30) Foreign Application Priority Data
Jul. 26, 2021 (IN) 202121033510

(51) Int. Cl. A61K 31/522; A61K 9/20; A61K 9/24; A61K 9/28; A61K 31/7034

(52) U.S. Cl. CPC A61K 31/522; A61K 9/2013; A61K 9/2018; A61K 9/2054; A61K 9/209; A61K 9/284; A61K 31/7034

(57) ABSTRACT

The present invention relates to a stable pharmaceutical composition comprising combination of a therapeutically effective amount of Sodium-glucose co-transporter-2 (SGLT2) inhibitor and a therapeutically effective amount of Dipeptidyl peptidase-4 (DPP4) inhibitor for reducing blood glucose level in patients with diabetes. The present invention specifically relates to a stable pharmaceutical composition comprising Dapagliflozin or a pharmaceutically acceptable salts or solvates thereof, and Linagliptin or a pharmaceutically acceptable salts or solvates thereof in presence of one or more pharmaceutically acceptable excipients. Moreover, the present invention relates to a stable pharmaceutical composition comprising Dapagliflozin. Linagliptin and one or more pharmaceutically acceptable excipients with reduced degradation impurities.

### PRIORITY APPLICATION

This application claims the benefit of priority of our Indian patent application 202121033510 filed on Jul. 26, 2021 which is incorporated herein by reference and disclosures of which is incorporated in the instant application.

### FIELD OF INVENTION

The present invention relates to a stable pharmaceutical composition comprising combination of a therapeutically effective amount of Sodium-glucose co-transporter-2 (SGLT2) inhibitor and a therapeutically effective amount of Dipeptidyl peptidase-4 (DPP4) inhibitor for reducing blood glucose level in patients with diabetes. The present invention specifically relates to a stable pharmaceutical composition comprising Dapagliflozin or a pharmaceutically acceptable salts or solvates thereof and Linagliptin or a pharmaceutically acceptable salts or solvates thereof in the presence of one or more pharmaceutically acceptable excipients. Moreover, the present invention relates to a stable pharmaceutical composition comprising Dapagliflozin, Linagliptin and one or more pharmaceutically acceptable excipients with reduced degradation impurities.

### BACKGROUND OF THE INVENTION

Diabetes mellitus (DM), commonly known as diabetes, is a group of metabolic disorders characterized by a high blood sugar level over a prolonged period of time due to deficiency in insulin secretion (Type 1 diabetes) or from resistance to insulin action combined with an inadequate insulin secretion (Type 2 diabetes). According to International Diabetes Federation (IDF), there are currently 537 million people living with diabetes in the world with Southeast Asia region accounting for 90 million people out of which 77 million people belong to India. 541 million adults are at increased risk of developing type 2 diabetes. Hence, there is huge requirement of anti-diabetic medications for treatment of diabetes.

Type 2 diabetes mellitus is a progressive disease wherein monotherapy alone cannot maintain glycemic control and leads to treatment failure. Usually, a combination of glucose-lowering agents with complementary mechanisms of action that can address multiple pathophysiologic pathways, can be used at all stages of the disease and are generally well tolerated with no increased risk of hypoglycemia, cardiovascular events or weight gain. The combination should also provide convenience for patients, such as oral dosing, single-pill formulations and once-daily administration, potentially translating to improved adherence. Two classes of glucose-lowering agents that meet these criteria are the sodium glucose cotransporter-2 (SGLT2) inhibitors and dipeptidyl peptidase-4 (DPP-4) inhibitors. Empagliflozin and Linagliptin, Dapagliflozin and Saxagliptin & Ertugliflozin and Sitagliptin combinations have been approved by USFDA as single pill formulations in the management of type 2 diabetes mellitus.

SGLT2 inhibitor is associated with glucosuria, an increase in the rate of endogenous glucose production (EGP), which offsets the glucose-lowering effect by approximately 50%. In contrast, DPP-4 inhibitor inhibits glucagon secretion and reduce EGP. This combination of DPP-4 inhibitor plus SGLT2 inhibitor would prevent the increase in EGP following SGLT2 inhibition and produce an additive and synergistic effect to reduce HbA1c.

SGLT2 inhibitors, also known as gliflozins, are a class of compounds that prevent the kidneys' reuptake of glucose from the glomerular filtrate and subsequently lower the glucose level in the blood and promote the excretion of glucose in the urine. SGLT2 is the major transport protein and promotes reabsorption from the glomerular filtration glucose back into circulation and is responsible for approximately 90% of the kidney's glucose reabsorption. Apart from blood sugar control, gliflozins have been shown to provide significant cardiovascular benefit in T2DM patients.

Dapagliflozin is a novel Sodium-glucose co-transporter-2 (SGLT2) inhibitor represented by following chemical structure.

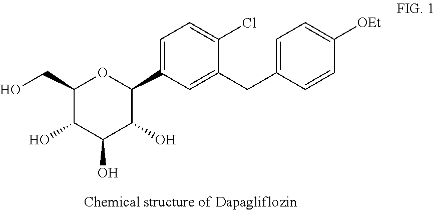 [SMILES: CCOc1ccc(Cc2cc([C@@H]3O[C@H](CO)[C@@H](O)[C@H](O)[C@H]3O)ccc2Cl)cc1]

The chemical name for Dapagliflozin is (1S)-1,5-Anhydro-1-[4-chloro-3-(4-methoxybenzyl)phenyl]-D-glucitol. It is developed by Bristol-Myers Squibb in partnership with AstraZeneca and is approved under different brand names for example, Forxiga®, Edistride® in Europe and Farxiga® in United States of America. Dapagliflozin is approved as 5 mg and 10 mg immediate release tablet dosage form for once daily administration for the treatment of insufficiently controlled type 2 diabetes mellitus, type 1 diabetes mellitus and heart failure. It is also under priority review for Chronic Kidney Disease (CKD) indication by USFDA.

U.S. Pat. No. 6,414,126 discloses markush structure covering Dapagliflozin, its analogous compounds and process for their preparation. U.S. Pat. No. 6,515,117 specifically discloses Dapagliflozin, its intermediates and process for their preparation.

U.S. Pat. No. 8,221,786 discloses the immediate release pharmaceutical composition of Dapagliflozin in the form of capsule or tablet or stock granules for loading in capsules or forming tablets containing one or more bulking agents, binders, disintegrants, glidants/anti-adherents and lubricants.

Dipeptidyl peptidase IV inhibitors, also known as gliptins, are a class of compounds that increase incretin levels which inhibits glucagon release which in turn increases insulin secretion and decreases blood glucose level.

Linagliptin is a novel Dipeptidyl peptidase-4 (DPP-IV) inhibitor represented by following chemical structure

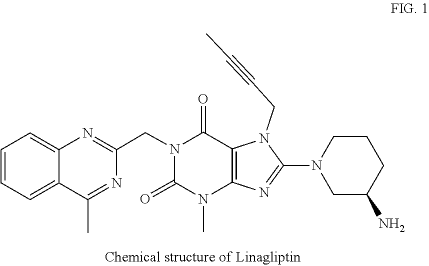 [SMILES: CC#CCn1c(N2CCC[C@@H](N)C2)nc2c1c(=O)n(Cc1nc(C)c3ccccc3n1)c(=O)n2C]

The chemical name for Linagliptin is 8-[(3R)-3-Aminopiperidin-1-yl]-7-(but-2-yn -1-yl)-3-methyl-1-[(4-methylquinazolin-2-yl)methyl]-3,7-dihydro-1H-purine-2,6-dione. It is developed by Boehringer Ingelheim and is approved under different brand names for example, Trajenta® in Europe and Tradjenta® in United States of America. Linagliptin is approved as 5 mg immediate release tablet dosage form for once daily administration an adjunct to diet and exercise to improve glycemic control in adults with type 2 diabetes mellitus.

U.S. Pat. No. 7,407,955 discloses Linagliptin or stereoisomers or pharmaceutically acceptable salts and its pharmaceutical composition. U.S. Pat. No. 11,033,552 discloses a tablet comprising Linagliptin or pharmaceutically acceptable salts thereof and a first diluent, a second diluent, a binder, a disintegrant and a lubricant, wherein the first diluent is mannitol, the second diluent is pregelatinized starch, the binder is copovidone, the disintegrant is corn starch, and the lubricant is magnesium stearate; and wherein the DPP IV inhibitor compound is present in an amount 0.5-7.0% on the total weight of DPP IV inhibitor compound, first diluent, second diluent, binder, disintegrant and lubricant.

WO 2009022010A1 discloses use of combination of SGLT2 inhibitor and DPP-IV inhibitor for the treatment of conditions such as diabetes and diseases related to diabetes. It discloses combination of Dapagliflozin (0.5 to 1000 mg) and Linagliptin (0.1 to 100 mg). However, it does not disclose pharmaceutical fixed dose combination for these two drugs.

WO 2019221473A1 discloses composition comprising Dapagliflozin and Linagliptin along with one or more excipients selected from mannitol, pregelatinized starch, microcrystalline cellulose, low-substituted hydroxypropyl cellulose, calcium phosphate, calcium carbonate and microcrystalline microcrystalline cellulose. However, it does not disclose anything about in-vivo synergistic or additive effect of this combination and level of impurities in final composition.

Yiwen Huang et al, J. Sep. Sci., 41(21), 3985-3994 discloses identification, characterization and quantification of impurity I which was detected during stress testing of Linagliptin under acidic condition. This unknown impurity I has structural genotoxic alert. However, the exact amount of impurity in finished dosage form during shelf-life is not known.

DPP-4 inhibitor, such as Linagliptin, with primary or secondary amino group tend to possess incompatibilities, degradation problems with many excipients including, but are not limited to, lactose, glucose, sucrose, fructose, saccharose, maltodextrin, cellulose derivatives such as microcrystalline cellulose, croscarmellose sodium. The primary or secondary amino group of these drug molecules tend to react with reducing sugars or with reactive carbonyl or carboxylic acid functional group associated with partner drug or its impurities or with many excipients and form N-acetyl or N-carbamoyl impurities many of which have genotoxic alert.

The above prior arts and marketed products disclose product administration of Dapagliflozin and Linagliptin into the patients suffering from diabetes mellitus. None of the compositions or combinations disclosed in prior art discuss about potential genotoxic impurities when linagliptin and dapagliflozin combined together in the formulation. In monotherapy treatment, different drug product needs to be administered in multiple times which is a burdensome and less compliant for the patients. Further, there are high chances of occurrence for medication errors due to multiple individual drug product administration and this seems to be less adherent treatment option for longer period of time.

The inventors of the present invention have overcome problems associated with the currently marketed products and have developed patient compliant and stable pharmaceutical compositions containing combination of Dapagliflozin or a pharmaceutically acceptable salts thereof and Linagliptin or a pharmaceutically acceptable salts thereof in single product with reduced amount of impurities. The Dapagliflozin and Linagliptin combination product would facilitate long unmet requirement of dosing regimen and instead of taking two products separately, patients can be treated with single product. This also leads to increase in patient adherence considering longer period treatment option and can reduce the pill burden. Moreover, there is an unaddressed issue of potential genotoxic impurity in such composition which have been overcome by the present invention composition with use of stabilizers.

### SUMMARY OF THE INVENTION

The present invention relates to a stable pharmaceutical composition comprising combination of a therapeutically effective amount of SGLT-2 inhibitor and a therapeutically effective amount of DPP-IV inhibitor for the treatment of diabetes mellitus. In one aspect of the invention, there is provided a pharmaceutical composition comprising a fixed dose combination of SGLT2 inhibitor and DPP-IV inhibitor or salt thereof and one or more pharmaceutically acceptable excipients.

The present invention specifically relates to a stable pharmaceutical composition comprising Dapagliflozin or a pharmaceutically acceptable salt thereof, Linagliptin or a pharmaceutically acceptable salt thereof and one or more pharmaceutically acceptable excipients.

The present invention specifically relates to a stable pharmaceutical composition comprising Dapagliflozin or a pharmaceutically acceptable salt thereof, Linagliptin or a pharmaceutically acceptable salt thereof and pharmaceutically acceptable stabilizers.

In still another aspect of the invention, there is provided a pharmaceutical composition comprising: (a) a first portion comprising Dapagliflozin or a pharmaceutically acceptable salt thereof and a pharmaceutically acceptable excipients and (b) a second portion comprising Linagliptin or a pharmaceutically acceptable salt thereof and a pharmaceutically acceptable excipients wherein the composition is a bilayer tablet. Optionally, the two layers in bilayer tablet are separated by barrier layer.

In still another aspect of the invention, there is provided a pharmaceutical composition comprising about 1 mg to 100 mg, even more preferably from about 1 mg to 50 mg, most preferably from about 1 mg to 10 mg of Dapagliflozin or a pharmaceutically acceptable salt thereof and about 1 mg to about 100 mg, even more preferably from about 1 mg to 50 mg, even more preferably from about 1 mg to about 10 mg DPP-IV inhibitor or a pharmaceutically acceptable salt thereof.

In another aspect of the invention, there is provided a pharmaceutical composition comprising a fixed dose combination of about 5 mg/10 mg of Dapagliflozin or equivalent amount of its salt or solvate thereof and about 2.5 mg/5 mg Linagliptin or equivalent amount of its salt or solvate thereof.

In another embodiment, the pharmaceutical composition of the present invention comprises one or more stabilizer along with Linagliptin part of the composition to limit the generation of impurity.

In particular, use of acidic compound, basic compound, amino acid, antioxidant and the likes as stabilizer in this composition helped to minimize the reaction of free amino group with reducing sugars or with reactive carbonyl or carboxylic acid functional group associated with partner drug or its impurities or with many excipients and provided composition with improved stability and reduced impurities.

The present invention specifically relates to a stable pharmaceutical composition comprising Dapagliflozin or a pharmaceutically acceptable salt thereof, Linagliptin or a pharmaceutically acceptable salt thereof and one or more pharmaceutically acceptable excipients, wherein the dapagliflozin is present either in immediate release or extended release composition form and linagliptin is also present either in immediate release or extended release composition form.

The present invention further relates to pharmaceutical composition for oral administration comprising Dapagliflozin or a pharmaceutically acceptable salt thereof and Linagliptin or a pharmaceutically acceptable salt thereof with not more than 0.4% w/w of unknown individual impurity for Linagliptin part.

The present invention further relates to pharmaceutical composition for oral administration for treatment of diabetes mellitus comprising Dapagliflozin or a pharmaceutically acceptable salt thereof and Linagliptin or a pharmaceutically acceptable salt thereof and a suitable stabilizer, wherein the composition remains stable at 40° C.±2° C./75% RH±5% RH conditions for a time period of at least 1 month and wherein specified or unspecified individual Linagliptin impurity is not more than 3% w/w, preferably not more than 2% w/w, more preferably not more than 1% w/w, more preferably not more than 0.4% w/w and total Linagliptin impurity is not more than 3% w/w, preferably not more than 2% w/w.

More particularly in alternative embodiment, the present invention relates to an oral dosage form comprising Dapagliflozin and Linagliptin, and optionally, one or more pharmaceutically acceptable excipients. More preferably, a core comprising a therapeutically effective amount of Linagliptin and a surrounding part comprising a therapeutically effective amount of Dapagliflozin with minimum contact between Dapagliflozin and Linagliptin which provides unexpected increase in stability relative to unknown individual impurity.

The present invention further relates to pharmaceutical composition for oral administration for treatment of diabetes mellitus comprising Dapagliflozin or a pharmaceutically acceptable salt thereof and Linagliptin or a pharmaceutically acceptable salt thereof, wherein 90% of amount of Dapagliflozin and/or Linagliptin initially present remains after composition stored at 40° C.±2° C./75% RH±5% RH conditions for a time period of at least 6 months.

The present invention is directed to a solid pharmaceutical composition for oral administration comprising Dapagliflozin or a pharmaceutically acceptable salt thereof and Linagliptin or a pharmaceutically acceptable salt thereof, in admixture with one or more excipients, more preferably a stabilizer, in a pharmacokinetically effective ratio such that said Dapagliflozin and said Linagliptin are released in a bioequivalent manner.

In still another embodiment, the formulation may be a film-coated tablet in which Dapagliflozin is present in the core tablet and Linagliptin is present in the film-coating layer along with one or more stabilizer. Alternatively, the formulation may be a barrier-coated tablet wherein Dapagliflozin and Linagliptin are not in direct physical contact with each other or they are present in direct contact with each other wherein surface area of their direct physical contact is minimized to increase the stability of the formulation. Alternatively, the tablet may be a trilayer tablet in which the two layers containing only Dapagliflozin and Linagliptin are separated by a third layer which does not contain any active ingredient. Alternatively, the tablet may be a press-coated tablet, i.e. a tablet in which Dapagliflozin is contained in small tablets and the Linagliptin is contained in a second granulation or blend and compressed together with one small tablet to one large press-coated tablet or via versa. All types of the herein before mentioned tablets may be without a coating or may have one or more coatings, in particular film-coatings.

In still another aspect of the invention, there is provided a pharmaceutical composition comprising: (a) an intra-granular portion comprising Dapagliflozin or a pharmaceutically acceptable salt thereof and a pharmaceutically acceptable excipients and (b) an extra-granular portion comprising Linagliptin or a pharmaceutically acceptable salt thereof or vice versa and a pharmaceutically acceptable excipients. Alternatively, Dapagliflozin or a pharmaceutically acceptable salt thereof and Linagliptin or a pharmaceutically acceptable salt thereof can be either in extragranular part compressed with inert core.

The present invention further relates to a process of preparing pharmaceutical composition comprising the steps of: (i) blending Dapagliflozin or a pharmaceutically acceptable salt thereof and at least one pharmaceutically acceptable excipient or preparing granules; (ii) blending Linagliptin or a pharmaceutically acceptable salt thereof and at least one pharmaceutically acceptable excipient or preparing granules; (iii) blending the mixture or granules obtained in step (i) with the mixture obtained in step (ii) and at least one pharmaceutically acceptable excipient; (iv) finally formulating the mixture obtained in step (iii) into suitable pharmaceutically acceptable dosage form.

In further aspect of the invention, the pharmaceutical composition according to present invention is used for prevention, treatment or prophylaxis of diabetes. Further, there is provided a use of kit comprising the pharmaceutical composition according to present invention, for prevention, treatment or prophylaxis of diabetes.

### DETAILED DESCRIPTION

The present invention relates to a stable pharmaceutical composition comprising combination of a therapeutically effective amount of SGLT-2 inhibitor and a therapeutically effective amount of DPP-IV inhibitor for the treatment of diabetes mellitus.

Specifically, the present invention relates to a stable pharmaceutical composition comprising a therapeutically effective amount of Dapagliflozin or a pharmaceutically acceptable salt thereof, a therapeutically effective amount of Linagliptin or a pharmaceutically acceptable salt thereof and one or more pharmaceutically acceptable excipients.

The present invention more specifically relates to a stable pharmaceutical composition comprising Dapagliflozin or a pharmaceutically acceptable salt or solvate thereof, Linagliptin or a pharmaceutically acceptable salt thereof and pharmaceutically acceptable stabilizers.

Furthermore, the present invention relates to a pharmaceutical composition comprising: (a) a first portion comprising Dapagliflozin or a pharmaceutically acceptable salt thereof and a pharmaceutically acceptable excipients and (b) a second portion comprising Linagliptin or a pharmaceutically acceptable salt thereof and a pharmaceutically acceptable excipients wherein the composition is a bilayer tablet. Optionally, the two layers in bilayer tablet are separated by barrier layer.

As used herein, the term “Dapagliflozin” refers to compound which may be present in base form or in the form of the pharmaceutically acceptable salt or solvate or prodrug or metabolite or analog or isomer or like thereof.

As used herein, the term “Linagliptin” refers to compound which may be present in its base form or any of its pharmaceutically acceptable salt or solvate or prodrug or metabolite or analog or isomer or like thereof.

As used herein, the term “pharmaceutically acceptable salts” include, but are not limited to mineral or organic salts of basic residues such as amines, alkali or mineral or organic salts of acidic residues such as carboxylic acids and the like thereof. Further, the pharmaceutically acceptable salts include the conventional non-toxic salts or the quaternary ammonium salts. The conventional non-toxic salts include inorganic or organic acids, for example those derived from inorganic acids such as hydrochloric, hydrobromic, sulfuric, sulfamic, phosphoric, nitric and the like thereof; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isethionic, and the like thereof.

As used herein, the term “therapeutically effective amount of Dapagliflozin” is an amount of Dapagliflozin or its pharmaceutically acceptable salt which eliminates, alleviates, or provides relief in diabetes mellitus.

As used herein, the term “therapeutically effective amount of Linagliptin” is an amount of Linagliptin or its pharmaceutically acceptable salt which eliminates, alleviates, or provides relief in diabetes mellitus.

The terms “solid oral dosage form”, “oral dosage form”, “unit dose form”, “dosage form for oral administration” and the like are used interchangeably, and refer to a pharmaceutical composition in the form of a mini-tablets, pellets, pills, granules, beads, sachets, tablets in tablets, tablets in capsules, capsules, caplets, capsule, gelcap, geltab, pill, dry syrup, suspension or the like dosage forms.

The term “immediate release (IR)” refers to the drug to dissolve in the gastrointestinal contents, with no intention of delaying or prolonging dissolution or absorption of drug.

The term “extended release (ER)” refers to drug the formulation which makes the drug available over an extended period after ingestion. This allows a reduction in dosing frequency compared to a drug presented as a conventional dosage form (e.g., as a solution or an immediate release dosage form). In particular the extended release system may include, but are not limited to, swelling-controlled system, erosion-controlled system or diffusion-controlled system.

The term “stable” means a drug substance and/or pharmaceutical composition for pharmaceutical use which remains stable as per ICH guidelines.

The term “ICH guidelines” means drug substance and composition remains stable for longer period of time at 25° C.±2° C./60% RH±5% RH, 30° C.±2° C./65% RH±5% RH, and 40° C.±2° C./75% RH±5% RH conditions for a period of at least 6 months.

As used herein, the terms “about” and “approximately” should be understood to mean within an acceptable range for the particular value as determined by one of ordinary skill in the art, which will depend in part on how the value is measured or determined, i.e., the limitations of the measurement system. For example, “about” can mean a range of up to 30%, preferably up to 20%, preferably up to 10%, more preferably up to 5%, and more preferably still up to 1% of a given value.

As used herein, the terms “bioequivalence” is defined as a pharmacokinetic (PK) comparison of the present pharmaceutical composition to that of the approved formulation. The pharmaceutical composition of the present invention must display drug pharmacokinetics that fall within a range of 80-125% (0.8-1.25) when one computes the ratio of the drug PK of the present invention composition with respect to approved marketed formulation. The PK parameters that are used for this comparison are the maximum concentration achieved in the blood (Cₘₐₓ) and the area-under-the-curve (AUC). The AUC is determined by plotting the concentration of the active ingredient in the blood over time. It is accepted as bioequivalent if the present invention composition PK falls within the 80-125% range when compared to the approved marketed drug formulation PK.

As used herein, the term “impurity” include total impurities or individual impurities.

In one embodiment, the SGLT-2 inhibitor compound is selected from the group consisting of Dapagliflozin, Empagliflozin, Canagliflozin, Ertugliflozin, Sotagliflozin, Luseogliflozin, Tofogliflozin, Remogliflozin Etabonate, Ipragliflozin, or a like thereof.

In another embodiment, the DPP-IV inhibitor compound is selected from the group consisting Alogliptin, Vildagliptin, Saxagliptin, Linagliptin, Sitagliptin, Gemigliptin, Anagliptin, Teneligliptin, Trelagliptin, Omarigliptin, Evogliptin, Gosogliptin, Dutagliptin, Melogliptin, Denagliptin or a like thereof.

According to one aspect, there is provided a stable pharmaceutical composition comprising Dapagliflozin and Linagliptin wherein Dapagliflozin and Linagliptin are present in a ratio in the range from about 2:1 to about 4:1. In a preferred embodiment, Dapagliflozin and Linagliptin in present invention are present in a ratio of about 2:1.

In another aspect of the invention, there is provided a stable pharmaceutical composition comprising Dapagliflozin or a pharmaceutically acceptable salt thereof, Linagliptin or a pharmaceutically acceptable salt thereof and one or more pharmaceutically acceptable excipients, wherein the Dapagliflozin is present either in immediate release or extended release composition form and Linagliptin is also present either in immediate release or extended release composition form.

According to second aspect, the Dapagliflozin or its pharmaceutically acceptable salt thereof is present in the pharmaceutical composition in the range from about 1 mg to about 100 mg, preferably from about 1 mg to about 50 mg and more preferably from about 1 mg to about 10 mg.

According to third aspect, the Linagliptin or its pharmaceutically acceptable salt thereof is present in the pharmaceutical composition in the range from about 1 mg to about 100 mg, preferably from about 1 mg to about 50 mg and more preferably from about 1 mg to about 10 mg.

According to fourth aspect, the present invention is directed to a solid pharmaceutical composition for oral administration which comprises Dapagliflozin or a pharmaceutically acceptable salt thereof and Linagliptin or a pharmaceutically acceptable salt thereof, in admixture with one or more excipients, in a pharmacokinetically effective ratio such that said Dapagliflozin and said Linagliptin are released in a bioequivalent manner. In a particular embodiment, the Dapagliflozin and Linagliptin are released from said formulation simultaneously, at a rate and in a ratio providing each in a therapeutically effective and non-toxic amount.

According to fifth aspect, the pharmaceutical composition for oral administration comprises about 1-10 mg Dapagliflozin and 1-10 mg Linagliptin respectively.

In one embodiment, the present invention relates to pharmaceutical composition comprising Dapagliflozin, Linagliptin and one or more pharmaceutically acceptable excipients. In another embodiment, the present invention relates to the method for administration of pharmaceutical composition comprising Dapagliflozin, Linagliptin and one or more pharmaceutically acceptable excipients by administering in once daily oral dosage form.

The pharmaceutical compositions are present in any one of the unit dose form such as tablets, mini-tablets, pellets, pills, granules, beads, sachets, tablets in tablets, tablets in capsules, capsules, caplets, dry syrup, suspension or a like thereof.

The present invention addresses the problems associated with the current therapy of Dapagliflozin and Linagliptin. Currently, Dapagliflozin and Linagliptin products are needed to be administered individually into the patients suffering from diabetes mellitus which seems to be burdensome and less complaint therapy for the patients.

Further, there are high chances of occurrence for medication errors due to multiple individual drug product administration and this seems to be less adherent treatment option for longer period of time.

The inventors of the present invention have developed Dapagliflozin and Linagliptin combination pharmaceutical composition which would facilitate long unmet requirement of dosing regimen and instead of taking two products separately, patients can be treated with single product. This also leads to increase in patient adherence considering longer period treatment option and can reduce the pill burden. Further, combination therapy administration may provide synergistic effects and superior efficacy in comparison to monotherapy.

In one embodiment, the present invention relates to the pharmaceutical compositions comprising Dapagliflozin having a particle size D₉₀ less than 200 μm. The particle size D₉₀ preferred for use in the present invention is less than 150 μm, more preferably less than 100 μm and even more preferably less than 50 μm.

In another embodiment, the present invention relates to the pharmaceutical compositions comprising Linagliptin having a particle size D₉₀ less than 150 μm.

The particle size D₉₀ preferred for use in the present invention is less than 100 μm, more preferably less than 75 μm and even more preferably less than 50 μm.

The active pharmaceutical ingredient in the present invention composition may present in any one polymorphic form selected from crystalline, amorphous, hydrated, anhydrous or a like thereof.

In another embodiment, the present invention relates to the pharmaceutical composition comprising Dapagliflozin, Linagliptin and one or more pharmaceutically acceptable excipient wherein the composition remains stable at 25° C.±2° C./60% RH±5% RH, 30° C.±2° C./65% RH±5% RH, and 40° C.±2° C./75% RH±5% RH conditions for a time period of at least 6 months.

In another embodiment, there is no significant change in content level of Dapagliflozin and Linagliptin in the said pharmaceutical composition after 6 months storage stability conditions at 40° C.±2° C./75% RH±5% RH, where total known and unknown impurities level remain in the limit as per the defined criteria of USP/EP monographs.

Specifically, the total known and unknown impurities level for Linagliptin may range up to about 3.0% w/w in the composition, preferably less than about 2.7% w/w in the present invention. The individual unknown impurities present in an amount of less than about 0.4% w/w in the compositions.

In one embodiment, the present invention relates to the pharmaceutical composition comprising from about 1% to about 10% w/w of Dapagliflozin, from about 1% to about 10% w/w Linagliptin, and one or more pharmaceutically acceptable excipients.

In another embodiment, the pharmaceutical composition of the invention may include one or more pharmaceutically acceptable excipients selected from diluents, binders, disintegrants/superdisintegrants, lubricants, glidants, coloring agents, stabilizers, solvents, suitable film-forming agents or a like thereof.

The diluents according to the present invention include, but are not limited to, starch (maize starch, potato starch, rice starch, wheat starch, pregelatinized starch and others), lactose (e.g., lactose monohydrate, such as Fast Flo® 316, lactose anhydrous and others), cellulose derivatives includes crystalline celluloses such as microcrystalline cellulose, kaolin and powdered celluloses, confectioner's sugar, calcium carbonate, magnesium carbonate, dibasic calcium phosphate, tribasic calcium phosphate, dicalcium phosphate, calcium sulfate, carmellose, sugar alcohols such as mannitol, sorbitol, xylitol, inositol sucrose, inositol, polysaccharides polymers such as pullulan and mixtures thereof. Preferably, the diluent in present invention is mannitol and lactose. The diluent may present in an amount from about 30% to about 90% w/w of the composition, preferably from about 40% to about 80% w/w of the composition.

The binders according to the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone and its derivatives in various grades such as povidone, copovidone and others, methyl cellulose, pre-gelatinized starch, hydroxypropyl methyl cellulose in various grades, microcrystalline cellulose and mixtures thereof. Preferably, the binder in present invention is hydroxypropylmethylcellulose and povidone. The binder may present in an amount from about 0.5% to about 10% w/w of the composition, preferably from about 1.0 to about 5% w/w of the composition and more preferably from about 2% to about 4% % w/w of the composition.

The disintegrants/superdisintegrants according to the present invention include, but are not limited to, low-substituted hydroxypropyl cellulose, microcrystalline cellulose, sodium starch glycolate, alginic acid, calcium carbonate, croscarmellose sodium, crospovidone, polacrilin potassium, potato or tapioca starch, pre-gelatinized starch and mixtures thereof. The disintegrant present either alone or in combination with other disintegrants and the preferred disintegrant is croscarmellose sodium and low-substituted hydroxypropyl cellulose. The disintegrant may present in amount from about 1 to about 10% w/w of the composition, preferably from about 1 to about 5% w/w of the composition and more preferably about 2% w/w of the composition.

The lubricants according to the present invention include, but are not limited to, magnesium stearate, glyceryl monostearates, glyceryl behenate, palmitic acid, talc, carnauba wax, calcium stearate, zinc stearate, polyoxyethylene monostearates, calcium silicate, silicon dioxide, hydrogenated vegetable oils and fats, stearic acid, sodium stearyl fumarate and mixtures thereof. Preferably, the lubricant in present invention is magnesium stearate. The lubricants may present in an amount from about 0.01 to about 5% w/w of the composition, preferably from about 0.5 to about 2% w/w of the composition and more preferably about 1% w/w of the composition.

The glidants according to the present invention include, but are not limited to, silica such as colloidal silicon dioxide or kaolin, talc and mixtures thereof. Preferably, the glidant in present invention is colloidal silicone dioxide. The glidants may present in amount from about 0.01% to about 5% w/w of the composition, preferably from about 0.1% to about 1% w/w of the composition.

The coloring agents according to the present invention include, but are not limited to, natural colorants, synthetic colorants or like thereof. Examples of natural colorants include pigments and dyes obtained from mineral, plant, and animal sources like red ferric oxide, titanium dioxide, yellow ferric oxide, zinc oxide, indigo and synthetic colorants include FD&C or D&C dye, an azo dye or a like thereof. The colouring agents may be present in the composition as per the quantity sufficient requirement, preferably from about 0.1% to about 1.0 w/w of the composition.

The stabilizers may be included in the compositions of the present invention depending upon the requirement which include, but are not limited to acidic compound selected from the group comprising acetic acid, glacial acetic acid, citric acid, fumaric acid, hydrochloric acid, malic acid, nitric acid, phosphoric acid, propionic acid, sulfuric acid, tartaric acid, benzoic acid, sodium benzoate and the like or basic compound selected from the group comprising basic amino acids such as L-arginine, L-lysine, L-histidine, L-citrulline, cysteine, 6-amino caproic acid and the like or basic/alkalizing agents selected from the group comprising ammonium carbonate, sodium carbonate, monoethanolamine, diethanolamine, potassium hydroxide, sodium hydroxide, potassium carbonate and the like or buffering agents selected from the group comprising acetic acid, adipic acid, ammonium carbonate, ammonium phosphate, boric acid, citric acid, lactic acid, phosphoric acid, potassium citrate, potassium metaphosphate, potassium phosphate monobasic, potassium phosphate dibasic, sodium acetate, sodium citrate, sodium lactate, sodium phosphate monobasic, sodium phosphate dibasic, succinic acid and the likes. The stabilizer may further include antioxidant substances which is present in amounts effective to retard decomposition of a drug that is susceptible to oxidation. The antioxidants according to the present application include, but are not limited to include one or more of ascorbic acid and its salts, tocopherols, sulfite salts such as sodium metabisulfite or sodium sulfite, sodium sulfide, butylated hydroxyanisole, butylated hydroxytoluene, ascorbyl palmitate, propyl gallate and mixtures thereof. Any other category excipient may be included in the stabilizer list in the present invention pharmaceutical composition. The stabilizers may be present in amount from about 0.001 to about 10% w/w of the composition.

The solvents according to the present invention, but are not limited to, for the purpose of film coating/granulation includes water, methanol, ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulfoxide, N,N-dimethylformamide, tetrahydrofuran, and any mixtures thereof.

The compositions of the present invention may be coated with one or more film forming materials as known in the art. These coatings may be sugar coatings, film coatings, color coatings, drug coating or the like thereof.

The film-forming agents according to the present invention includes, but are not limited to, a water-soluble/water-insoluble film forming polymers, such as hydroxpropyl methylcellulose, methylcellulose, ethylcellulose, hydroxypropyl cellulose, povidone, polydextrose, lactose, maltodextrin, acrylic polymer such as ammonium ethacrylate copolymers, polyvinyl derivative, polyvinyl acetate, Poly (ethylene oxide), Poly (ethylene glycol) and mixtures thereof. The film coating may optionally contain a plasticizer, such as castor oil, polyethylene glycol, propylene glycol or glycerine, and a coloring or pacifying agent. The film coating may also contain a flavoring and/or sweetening agent to improve palatability. A preferred blend of hydroxypropyl methylcellulose, a plasticizer and a colorant is commercially available from Colorcon Company under the trade name OPADRY®.

According to one embodiment, the pharmaceutical composition is present in any one of the form selected from single layer composition, bi-layer composition, multi- layer composition or any other alternative modification in the composition as accompanied in the present invention.

The dosage forms herein, e.g., fixed dose combination tablets, can be of any suitable size and shape and the invention is not limited in this regard. For example, the dosage forms may be of triangular, round, rectangular, square, capsule, almond, oval, diamond, biconvex, multi-layered, or have an irregular shape. There may also be letters or characters embossed or printed on the dosage form surface.

In another embodiment, the present invention relates to a pharmaceutical composition comprising Dapagliflozin or a pharmaceutically acceptable salt or solvate thereof in an amount from about 1.0% to about 10% w/w, Linagliptin or a pharmaceutically acceptable salt thereof in an amount from about 1.0% to about 10% w/w, diluent in an amount from about 30% to about 90% w/w, a disintegrant in an amount from about 0% to about 10% w/w, a binder in an amount from about 0% to about 10% w/w, lubricant in an amount from about 0.1% to about 5% w/w, a colouring agent in an amount from about 0% to about 1% w/w, glidant in an amount from about 0.1% to about 5% w/w, optionally a stabilizer in an amount from about 0.001% to about 10% w/w and film forming substance in an amount from about 0% to about 10% w/w of the composition.

In another embodiment, the present invention relates to a stable pharmaceutical composition comprising, Dapagliflozin propanediol monohydrate, Linagliptin, microcrystalline cellulose, stabilizer selected from group comprising of L-arginine, ascorbic acid, butylated hydroxy tolene, butylated hydroxyl anisole or a combination thereof, zinc stearate, colloidal anhydrous silica and optionally film forming agent.

In another embodiment, the present invention relates to a pharmaceutical composition comprising, of total composition, about 4.1% w/w Dapagliflozin propanediol monohydrate, about 1.67% w/w Linagliptin, about 90% w/w microcrystalline cellulose, about 0.01% to 5% w/w stabilizer selected from the group comprising of L-arginine, ascorbic acid, butylated hydroxy tolene, butylated hydroxyl anisole or a combination thereof, about 1% w/w zinc stearate, about 1% w/w colloidal anhydrous silica and optionally about 4% w/w film forming agent.

In another embodiment, the present invention relates to a pharmaceutical composition comprising, about 12.3 mg Dapagliflozin propanediol monohydrate, about 5.00 mg Linagliptin, about 275.2 mg microcrystalline cellulose, stabilizer selected from group comprising of about 6.00 mg L-arginine, about 3.00 mg ascorbic acid, about 1.5 mg butylated hydroxy tolene, about 1.5 mg butylated hydroxyl anisole or a combination thereof, about 3.00 mg zinc stearate, about 3 mg colloidal anhydrous silica and optionally about 12.00 mg film forming agent.

In another embodiment, the present invention relates to a bilayer pharmaceutical composition Dapagliflozin, Linagliptin, microcrystalline cellulose as a diluent, lactose monohydrate as a diluent, hydroxypropyl cellulose as disintegrant, hydroxypropylcellulose as a binder, zinc stearate as a lubricant, colloidal silicon dioxide as glidant and optionally polyvinyl alcohol based film coating substance (Opadry) wherein the first layer comprises—Linagliptin, microcrystalline cellulose, hydroxypropylmethylcellulose, zinc stearate and the second layer comprises—Dapagliflozin, microcrystalline cellulose, lactose monohydrate, hydroxypropylcellulose, colloidal anhydrous silica, iron oxide yellow and Zinc stearate.

In another embodiment, the present invention relates to a bilayer pharmaceutical composition comprising, of total composition, about 2.77% w/w Dapagliflozin propanediol monohydrate, about 1.13% w/w Linagliptin, about 52.25% w/w microcrystalline cellulose, about 34.00% w/w lactose, about 2.82 w/w hydroxyl propyl cellulose, about 1.8% hypromellose, about 0.03% w/w iron oxide yellow, about 1% w/w zinc stearate, about 1.2% w/w colloidal anhydrous silica and optionally about 3% w/w film forming agent.

In another embodiment, the present invention relates to a bilayer pharmaceutical composition comprising, about 12.3 mg Dapagliflozin propanediol monohydrate, about 5 mg Linagliptin, about 232.00 mg microcrystalline cellulose, about 152.00 mg lactose, about 12.5 mg hydroxyl propyl cellulose, about 8.00 mg hypromellose, about 0.125 mg iron oxide yellow, about 3.8 mg zinc stearate, about 5.00 mg colloidal anhydrous silica and optionally about 12.9 mg film forming agent.

The pharmaceutical composition of the present invention can be obtained by a known conventional methods like direct compression, wet granulation, dry granulation, roller compaction or slugging, fluidized bed granulation, rapid mixture granulation, solvent evaporation, hot-melt extrusion or like thereof. The wet granulation process may involve shear granulators (such as planetary mixers), high shear mixer granulators (such as Fielder or Diosna), twin screw granulators (such as ConsiGma) and Fluid Bed Granulators (such as Aeromatic or Glatt).

According to one aspect, the pharmaceutical compositions can be prepared by a process comprising the steps of: (i) blending Dapagliflozin or a pharmaceutically acceptable salt thereof and at least one pharmaceutically acceptable excipient; (ii) blending Linagliptin or a pharmaceutically acceptable salts thereof and at least one pharmaceutically acceptable excipient; (iii) blending the mixture obtained in step (i) with the mixture obtained in step (ii) and at least one pharmaceutically acceptable excipient; (iv) finally formulating the mixture obtained in step (iii) into suitable pharmaceutically acceptable dosage form.

According to third aspect, the pharmaceutical compositions can be prepared by direct compression method comprising the steps of: i) preparing a powder mixture containing Dapagliflozin, Linagliptin and pharmaceutical excipients, and ii) subjecting the powder mixture obtained in step (i) to compression to obtain the tablet.

According to second aspect, the pharmaceutical compositions can be prepared by wet granulation method comprising the steps of: i) preparing a mixture containing Dapagliflozin, Linagliptin and at least one pharmaceutical excipient, ii) granulating the mixture obtained in step (i) with a granulation liquid to form a wet granulate, iii) drying the wet granulate obtained in step (ii) to form a dry granulate, iv) optionally mixing the granulate obtained in step (iii) with a pharmaceutical excipient, and v) subjecting the granulate obtained in step (iii) or the mixture obtained in step (iv) to compression to obtain the tablet.

According to fourth aspect, the pharmaceutical compositions can be prepared by dry granulation method comprising the steps of: i) preparing a powder mixture containing Dapagliflozin, Linagliptin and at least one pharmaceutical excipient, ii) compacting the powder mixture obtained in step (i) to form a compact/slug/ribbon, iii) converting the compact/slug/ribbon obtained in step (ii) into a granulate, iv) optionally mixing the granulate obtained in step (iii) with a pharmaceutical excipient, and v) subjecting the granulate obtained in step (iii) or the mixture obtained in step (iv) to compression to obtain the tablet.

According to fifth aspect, the pharmaceutical compositions can be prepared by fluid bed granulation a process comprising the steps of: (i) dissolving a mixture of Dapagliflozin, Linagliptin with one or more pharmaceutical excipients in suitable solvents, (ii) followed by solvent spray onto the powder for the granulation purpose, (iv) blending the obtained granules with extra-granular excipients and lubricating the blend, (v) at last compressing the blend obtained to form a tablet composition and (vi) optionally coating the said composition.

According to sixth aspect, the bi-layer pharmaceutical compositions can be prepared by direct compression method comprising the steps of: i) preparing a powder mixture containing Dapagliflozin and pharmaceutical excipients, and ii) preparing a powder mixture containing Linagliptin and pharmaceutical excipients, iii) subjecting the powder mixture obtained in step (i) and step (ii) to compression to obtain the tablet.

According to seventh aspect, the bi-layer pharmaceutical compositions can be prepared by granulation methods such as wet or dry granulation comprising the steps of: i) preparing a granules containing Linagliptin and pharmaceutical excipients by dry granulation or wet granulation methods, and ii) preparing a powder mixture containing Dapagliflozin or its salt or solvate thereof and pharmaceutical excipients, iii) subjecting the granules obtained in step (i) and powder mixture obtained in step (ii) to compression to obtain the tablet. Alternatively, the Dapagliflozin layer and Linagliptin layer prepared with different techniques, such as dry granulation and wet granulation, can also be compressed into tablet.

The pharmaceutical composition of the present invention can be preferably packed into blisters or bottles or a like thereof. Preferred blisters are made of material or laminate, which ensures high protection against humidity, oxygen and UV radiation. It is preferred that blisters are made of PVC, OPA (oriented polyamide), aluminium foil, PCTFE (e.g. Aclar), PVDC (polyvinylidene chloride), PVDC-coated PVC, PVC/PE/PCTFE laminate, CFF (Cold-Form Foil), COC (Cyclic Olefin Copolymer) or combination thereof.

According to one embodiment, the combination pharmaceutical composition of Dapagliflozin and Linagliptin exhibits bioequivalent plasma profile in comparison to marketed individual products of Dapagliflozin and Linagliptin. The pharmaceutical composition of the present invention can be used in the treatment of diabetes mellitus by administering effective amount of pharmaceutical compositions to the patient.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### REFERENCE EXAMPLE

TABLE 1
Single layer tablet of Dapagliflozin & Linagliptin

| Ingredients (mg/tab) | RU1 | RU2 |
|---|---|---|
| Dapagliflozin propanediol monohydrate | 12.3 | 12.3 |
| Linagliptin | 5.00 | 5.00 |
| Microcrystalline Cellulose | 264.7 | 254.7 |
| Microcrystalline Cellulose (Ceolus 1000 Kg) | 12.00 | 12.00 |
| Hypromellose | — | 13.00 |
| Purified water | — | q.s. |
| Colloidal anhydrous silica | 3.00 | — |
| Zinc Stearate | 3.00 | 3.00 |
| Total Weight | 300.00 | 300.00 |

Coating

| | | |
|---|---|---|
| Film forming agent (Opadry) | 12.00 | 12.00 |
| Purified water | q.s. | q.s. |
| Total weight of coated tablets | 312.00 | 312.00 |

### Preparation of RU1

1. Dapagliflozin propanediol monohydrate, Linagliptin, colloidal anhydrous silica and Microcrystalline Cellulose were sifted and mixed in blender.
2. Zinc stearate was sifted and mixed with the step 1 mixture to prepare blend for compression.
3. Step 2 blend was compressed into tablets.
4. Tablets obtained in step 3 were coated with film forming agent (Opadry).

### Preparation of RU2

1. Dapagliflozin propanediol monohydrate, Linagliptin and Microcrystalline Cellulose were sifted and dry mixed for 10 minutes.
2. Hypromellose was added to purified water and the mixture was stirred continuously to obtain clear solution.
3. Step 1 material was granulated using the solution obtained in step 2.
4. Granules obtained in step 3 were dried and sifted using suitable sieve.
5. Microcrystalline cellulose was sifted through suitable sieve and mixed with the dried granules obtained in step 4.
6. Zinc stearate was sifted through suitable sieve and mixed with step 5 mixture in a blender to prepare blend for compression.
7. Step 6 blend was compressed into tablets.
8. Tablets obtained in step 7 were coated with were coated with film forming agent (Opadry).

### EXAMPLES

### Example 1

TABLE 2
Single layer tablet of Dapagliflozin & Linagliptin with stabilizer

| Ingredients (mg/tab) | U1 | U2 | U3 | U4 |
|---|---|---|---|---|
| Dapagliflozin propanediol | 12.3 | 12.3 | 12.3 | 12.3 |
| monohydrate | | | | |
| Linagliptin | 5.00 | 5.00 | 5.00 | 5.00 |
| Microcrystalline Cellulose | 263.2 | 261.7 | 258.7 | 263.2 |
| Microcrystalline Cellulose (Ceolus | 12.00 | 12.00 | 12.00 | 12.00 |
| 1000 Kg) | | | | |
| Butylated hydroxy toluene (BHT) | 1.5 | — | — | — |
| Ascorbic acid | — | 3.00 | — | — |
| Butylated hydroxy anisole (BHA) | — | — | — | 1.5 |
| L-Arginine | — | — | 6.00 | — |
| Colloidal anhydrous silica | 3.00 | 3.00 | 3.00 | 3.00 |
| Zinc Stearate | 3.00 | 3.00 | 3.00 | 3.00 |
| Total Weight | 300.00 | 300.00 | 300.00 | 300.00 |

Coating

| | | | | |
|---|---|---|---|---|
| film forming agent (Opadry) | 12.00 | 12.00 | 12.00 | 12.00 |
| Purified water | q.s. | q.s. | q.s. | q.s. |
| Total weight of coated tablets | 312.00 | 312.00 | 312.00 | 312.00 |

### Manufacturing Procedure

1. Linagliptin, stabilizer (BHT/ Ascorbic acid/ L-Arginine/ BHA) and part quantity of microcrystalline cellulose were sifted through suitable sieves.
2. Dapagliflozin, part quantity of microcrystalline cellulose and colloidal anhydrous silica were sifted through suitable sieve.
3. Step 1 & step 2 materials were mixed in blender followed by mixing with remaining quantity of microcrystalline cellulose.
4. Zinc stearate was sifted through suitable sieve and mixed with step 3 mixture in a blender to prepare blend for compression.
5. Step 4 blend was compressed into tablets.
6. Tablets obtained in step 5 were coated with were coated with film forming agent (Opadry).

### Example 3: Stability Test

A stability test was performed to confirm the stability of the pharmaceutical composition of the present invention. Specifically, as shown in Table 3, the formulations of Example 1 were stored for 1 month under severe conditions (40° C./75% RH), and then the amount of impurities were evaluated.

TABLE 3
Evaluation of impurity in compositions of Example-1

| Impurity (%) | Stability | | | |
|---|---|---|---|---|
| (Linagliptin layer) | conditions | U1 | U2 | U3 |
| Unspecified Impurity | Initial | 0.048 | 0.049 | 0.092 |
| | 1M Alu/Alu | 0.050 | 0.054 | 0.106 |
| | (40° C./75% RH) | | | |
| Total Impurity | Initial | 0.300 | 0.301 | 0.440 |
| | 1M Alu/Alu | 0.241 | 0.293 | 0.284 |
| | (40° C./75% RH) | | | |

The stability test result shows that amount of impurities in formulation of Example-1 is within the limit.

### Example 2

TABLE 4
Bilayer Dapagliflozin + Linagliptin tablets

| | | |
|---|---|---|
| Ingredients (mg/tab) | U5 | % w/w |

First layer

| | | |
|---|---|---|
| Dapagliflozin propanediol monohydrate | 12.3 | 4.92% |
| Microcrystalline cellulose | 66.375 | 26.55% |
| Lactose Anhydrous | 151.7 | 60.68% |
| Low substituted hydroxy propyl cellulose | 12.5 | 5.00% |
| Iron Oxide Yellow | 0.125 | 0.05% |
| Colloidal anhydrous silica | 5.0 | 2.0% |
| Zinc Stearate | 2.0 | 0.8% |
| Weight of first layer tablets | 250 | 100% |

Second layer
Dry Mix

| | | |
|---|---|---|
| Linagliptin | 5.00 | 2.78% |
| Microcrystalline cellulose | 85.3 | 47.39% |

Binding

| | | |
|---|---|---|
| Hypromellose | 8.00 | 4.44% |
| Purified Water | q.s. | — |

Lubrication

| | | |
|---|---|---|
| Microcrystalline cellulose | 72.7 | 40.39% |
| Microcrystalline cellulose (Ceolus 1000 Kg) | 7.2 | 4% |
| Zinc Stearate | 1.8 | 1.00% |
| Weight of second layer tablets | 180 | 100% |
| Total Weight of tablets | 430 | — |

Coating composition

| | | |
|---|---|---|
| film forming agent (Opadry) | 12.9 | 2.9% |
| Purified water | q.s. | — |
| Total weight of coated tablets | 442.9 | — |

### Manufacturing Procedure

### A. First Layer Dapagliflozin:

1. Dapagliflozin propanediol monohydrate, part quantity of anhydrous lactose and colloidal anhydrous silica were sifted through suitable sieve.
2. Iron oxide Yellow (Sicovit yellow) was sifted through suitable sieve.
3. Step 1 and step 2 materials were mixed in blender.
4. Part quantity of anhydrous lactose, microcrystalline cellulose, and hydroxypropyl cellulose were sifted through suitable sieve.
5. Step 3 and step 4 materials were mixed in blender.
6. Zinc stearate was sifted through suitable sieve and mixed with step 5 mixture in a blender to prepare blend for compression.

### B. Second Layer Linagliptin:

7. Linagliptin and Microcrystalline Cellulose were sifted through sieves and mixed in blender.
8. Hypromellose was added to purified water and the mixture was stirred continuously to obtain clear solution.
9. Step 7 material was granulated using the solution obtained in step 8.
10. Granules obtained in step 9 were dried and sifted using suitable sieve.
11. Microcrystalline cellulose was sifted through suitable sieve and mixed with the dried granules obtained in step 10.
12. Zinc stearate was sifted through suitable sieve and mixed with step 11 mixture in a blender to prepare blend for compression.

### C. Compression:

13. First layer blend of step 6 and second layer blend of step 12 were compressed into bilayer tablets.

### D. Coating:

14. Bilayer tablets obtained in step 13 were coated with film forming agent (Opadry).

### Example 3: Stability Test

A stability test was performed to confirm the stability of the pharmaceutical composition of the present invention. Specifically, as shown in Table 5, the formulations of Example 2 were stored for 1 month under severe conditions (40° C./75% RH), and then the amount of impurities were evaluated.

TABLE 5
Evaluation of impurity in compositions of Example-2

| Impurity (%) | Stability | | | |
|---|---|---|---|---|
| (Linagliptin layer) | conditions | RU1 | RU2 | U5 |
| Unspecified | Initial | 0.048 | 0.095 | 0.062 |
| Impurity | 1M (40° C./75% RH) | 0.389 | 0.504 | 0.113 |
| | 1M Alu/Alu | 0.048 | 0.099 | 0.072 |
| | (40° C./75% RH) | | | |
| Total Impurity | Initial | 0.299 | 0.301 | 0.254 |
| | 1M (40° C./75% RH) | 1.282 | 1.586 | 0.690 |
| | 1M Alu/Alu | 0.205 | 0.245 | 0.411 |
| | (40° C./75% RH) | | | |

The stability test result shows that amount of impurities in bilayer formulation of Example-2 is within the limit compared to reference examples.

### Example 4: Dissolution Test

Comparative dissolution was performed for Linagliptin and Dapagliflozin for combination formulation of Examples 2 and the formulations of reference example 1. The dissolution test was performed with dissolution media containing hydrochloric Acid (HCl), water (pH 1.2, 900 mL) by using Apparatus 2 as described in Chapter 711 (Dissolution) of the US Pharmacopeia at 50 rpm at 37.0±0.5° C.

TABLE 6
Dissolution test results

Dissolution (%)
pH 1.2

| Time | | 15 min | 30 min | 45 min |
|---|---|---|---|---|
| RU1 | Dapagliflozin | 14 | 23 | 30 |
| | Linagliptin | 25 | 41 | 53 |
| RU2 | Dapagliflozin | 34 | 61 | 75 |
| | Linagliptin | 58 | 76 | 80 |
| U5 | Dapagliflozin | 79 | 86 | 89 |
| | Linagliptin | 88 | 98 | 101 |

The dissolution profile shows that the bilayer tablet composition of Dapagliflozin and Linagliptin had very superior release profile compared to reference examples.

1-10. (canceled)

11. A stable pharmaceutical composition comprising a combination of Linagliptin or pharmaceutically acceptable salt or solvate thereof with Dapagliflozin or pharmaceutically acceptable salt or solvate thereof and at least one pharmaceutically acceptable excipient.

12. The stable pharmaceutical composition of claim 11, wherein the pharmaceutical composition comprises a first portion and a second portion, wherein the first portion comprises Linagliptin or pharmaceutically acceptable salt or solvate thereof and a pharmaceutically acceptable excipient; and the second portion comprises Dapagliflozin or pharmaceutically acceptable salt or solvate thereof and a pharmaceutically acceptable excipient.

13. The stable pharmaceutical composition of claim 11, wherein the pharmaceutically acceptable excipient is selected from the group consisting of diluent, binder, disintegrant, stabilizer, lubricant, glidant, coloring agent, flavoring agents, coating agents, anti-tacking agents, and a combination thereof.

14. The stable pharmaceutical composition of claim 11, wherein the composition further comprises at least one pharmaceutically acceptable stabilizer.

15. The stable pharmaceutical composition of claim 14, wherein the stabilizer is selected from the group consisting of acidic agent, alkalizing agent, buffering agent, amino acid, anti-oxidant, and a combination thereof.

16. The stable pharmaceutical composition according to claim 11, wherein a weight ratio of Linagliptin or pharmaceutically acceptable salt or solvate thereof and of Dapagliflozin or pharmaceutically acceptable salt or solvate thereof ranges from about 1:4 to about 4:1.

17. The stable pharmaceutical composition according to claim 11, wherein the composition is in the form of a monolayer or a bilayer tablet.

18. The stable pharmaceutical composition according to claim 11, wherein the composition when stored at 40° C./75% relative humidity does not have more than 0.4% of individual impurity or more than 2% of total impurity.

19. The stable pharmaceutical composition according to claim 11, which is capable of releasing at least 75% of each of Dapagliflozin and Linagliptin within 45 minutes of ingestion.

20. The stable pharmaceutical composition according to claim 11, wherein the composition comprises 1.0-10% w/w Dapagliflozin propanediol monohydrate, 1.0-10% w/w Linagliptin, 30-90% w/w diluent, 0-10% w/w disintegrant, 0-10% w/w binder, 0.1-5% w/w lubricant, 0.1-5% w/w glidant, and optionally 0.001-10% w/w stabilizer.

21. The stable pharmaceutical composition of claim 12, wherein the pharmaceutically acceptable excipient is selected from the group consisting of diluent, binder, disintegrant, stabilizer, lubricant, glidant, coloring agent, flavoring agents, coating agents, anti-tacking agents, and a combination thereof.

22. The stable pharmaceutical composition according to claim 21, wherein a weight ratio of Linagliptin or pharmaceutically acceptable salt or solvate thereof and of Dapagliflozin or pharmaceutically acceptable salt or solvate thereof ranges from about 1:4 to about 4:1.

23. The stable pharmaceutical composition according to claim 12, wherein the composition is in the form of a monolayer or a bilayer tablet.

24. The stable pharmaceutical composition according to claim 12, wherein the composition when stored at 40° C./75% relative humidity does not have more than 0.4% of individual impurity or more than 2% of total impurity.
